(19) United States
(12) Patent Application Publication
(10) Pub. No.: US 2021/0158901 A1
(43) Pub. Date: May 27, 2021

(54) UTILIZING A NEURAL NETWORK MODEL AND HYPERBOLIC EMBEDDED SPACE TO PREDICT INTERACTIONS BETWEEN GENES

(71) Applicant: Accenture Global Solutions Limited, Dublin (IE)

(72) Inventors: Vivek Kumar KHETAN, San Francisco, CA (US); Maziyar BARAN POUYAN, Emeryville, CA (US)

(21) Appl. No.: 17/099,414

(22) Filed: Nov. 16, 2020

Related U.S. Application Data
(60) Provisional application No. 62/940,573, filed on Nov. 26, 2019.

(51) Int. Cl. G16B 40/20; G06N 3/08; G06N 3/04; G06N 3/12; G16B 5/20

(52) U.S. Cl. CPC G16B 40/20; G06N 3/08; G16B 5/20; G06N 3/126; G06N 3/04

(57) ABSTRACT

In some implementations, a prediction system may receive a gene regulatory network associated with genes. The prediction system may determine interactions between the genes associated with the gene regulatory network. The prediction system may generate a hyperbolic embedded space based on the gene regulatory network and the interactions between the genes. The prediction system may determine a hyperbolic distance measure based on the hyperbolic embedded space. The prediction system may process the hyperbolic embedded space and the hyperbolic distance measure, with a neural network model, to generate predictions of interactions between the genes. The prediction system may perform one or more actions based on the predictions of interactions between the genes.

### CROSS-REFERENCE TO RELATED APPLICATION

This Patent Application claims priority to U.S. Provisional Patent Application No. 62/940,573, filed on Nov. 26, 2019, and entitled “UTILIZING AN ARTIFICIAL INTELLIGENCE MODEL AND HYPERBOLIC SPACE TO PREDICT NEW INTERACTIONS BETWEEN GENES BACKGROUND.” The disclosure of the prior Application is considered part of and is incorporated by reference into this Patent Application.

### BACKGROUND

A gene regulatory network defines functional properties of genomic control programs (e.g., of an animal). The gene regulatory network includes nodes that represent genes and edges that represent physical and/or regulatory relationships between the genes. The gene regulatory network identifies interactions between the genes to manage molecular functions.

### SUMMARY

In some implementations, a method includes receiving, by a device, a gene regulatory network associated with genes; determining, by the device, interactions between the genes associated with the gene regulatory network; generating, by the device, a hyperbolic embedded space based on the gene regulatory network and the interactions between the genes; determining, by the device, a hyperbolic distance measure based on the hyperbolic embedded space; processing, by the device, the hyperbolic embedded space and the hyperbolic distance measure, with a neural network model, to generate predictions of interactions between the genes; and performing, by the device, one or more actions based on the predictions of interactions between the genes.

In some implementations, a device includes one or more memories; and one or more processors, communicatively coupled to the one or more memories, configured to: receive a gene regulatory network associated with genes; generate a hyperbolic embedded space based on the gene regulatory network; determine a hyperbolic distance measure based on the hyperbolic embedded space; process the hyperbolic embedded space and the hyperbolic distance measure, with a neural network model, to generate predictions of interactions between the genes; modify the gene regulatory network based on the predictions of interactions and to generate a modified gene regulatory network; and provide data identifying the modified gene regulatory network for display.

In some implementations, a non-transitory computer-readable medium storing a set of instructions includes one or more instructions that, when executed by one or more processors of a device, cause the device to: receive a gene regulatory network, wherein the gene regulatory network includes nodes that represent genes and edges between the nodes that represent relationships of the genes; generate a hyperbolic embedded space based on the gene regulatory network, wherein the hyperbolic embedded space includes a space in which data is embedded after dimensionality reduction; determine a hyperbolic distance measure based on the hyperbolic embedded space; process the hyperbolic embedded space and the hyperbolic distance measure, with a multi-layer deep neural network model, to generate predictions of interactions between the genes; and perform one or more actions based on the predictions of interactions between the genes.

### BRIEF DESCRIPTION OF THE DRAWINGS

FIGS. 1A-1E are diagrams of an example implementation described herein.

FIG. 2 is a diagram illustrating an example of training and using a machine learning model, such as a neural network model, in connection with predicting interactions between genes.

FIG. 3 is a diagram of an example environment in which systems and/or methods described herein may be implemented.

FIG. 4 is a diagram of example components of one or more devices of FIG. 3.

FIG. 5 is a flowchart of an example process relating to predicting interactions between genes.

### DETAILED DESCRIPTION

The following detailed description of example implementations refers to the accompanying drawings. The same reference numbers in different drawings may identify the same or similar elements.

A gene regulatory network defines functional properties of genomic control programs in animals, and includes a set of genes interacting with each other to manage molecular functions. A gene regulatory network may include nodes that represent the genes and edges that represent physical and/or regulatory relationships between the genes. A comprehensive study of gene interactions in a gene regulatory network enables researchers to identify functional relationships between genes and resulting proteins, as well as insights into underlying biological phenomena that are critical to understanding phenotypes in health and disease conditions. A topological structure of a gene regulatory network provides information for inferring functional patterns of genes (e.g., when researchers explore interactions of genes in the presence of various diseases in particular tissue).

Current solutions to determine interactions between genes are based on mapping a gene regulatory network to a linear Euclidean space. However, a linear Euclidean space is not able to completely represent a hierarchal inherent nature of interactions between genes since the linear Euclidean space is too narrow to embed hierarchical structures, loses information while representing gene hierarchy in latent space, creates difficult calculations of relative distances between genes, increases linearly and quadratically with regard to a number of dimensions, and/or the like.

Some implementations described herein provide a prediction system that utilizes a neural network model and hyperbolic embedded space to predict interactions between genes. In some implementations, the prediction system receives a gene regulatory network that includes nodes representing genes and edges representing relationships between the genes and determines interactions between the genes. The prediction system generates a hyperbolic embedded space (e.g., a non-Euclidean space in which data is embedded after dimensionality reduction, such as a Poincare embedded space based on a Poincare ball model) and determines a hyperbolic distance measure based on the hyperbolic embedded space. The hyperbolic embedded space and the hyperbolic distance measure indicate interactions that are unable to be represented in a linear Euclidean space. Accordingly, the prediction system utilizes the hyperbolic embedded space as input for a neural network model, such as a multi-layer deep neural network model, that generates predictions of interactions between genes in the gene regulatory network.

In this way, the prediction system utilizes a neural network model and hyperbolic embedded space to make predictions of interactions between genes. The predictions of interactions between genes in a gene regulatory network helps in understanding underlying biological phenomena that are critical to understanding phenotypes in health and disease conditions. The hyperbolic embedded space better captures a topological structure of the gene regulatory network (e.g., as compared to a linear Euclidean space), which aids in exploring interactions of genes in the presence of various diseases in particular tissue (e.g., tumors). Moreover, the hyperbolic embedded space captures similarity and hierarchy among genes, which is not possible with a linear Euclidean space.

Accordingly, the prediction system facilitates generation of predictions of interactions between genes that, in many cases, would not otherwise be found using conventional techniques associated with a linear Euclidean space. Therefore, the prediction system reduces a need for such conventional techniques and thereby reduces consumption of computing resources (e.g., processing resources, memory resources, communication resources, and/or power resources, among other examples) that would otherwise be used in association with the conventional techniques.

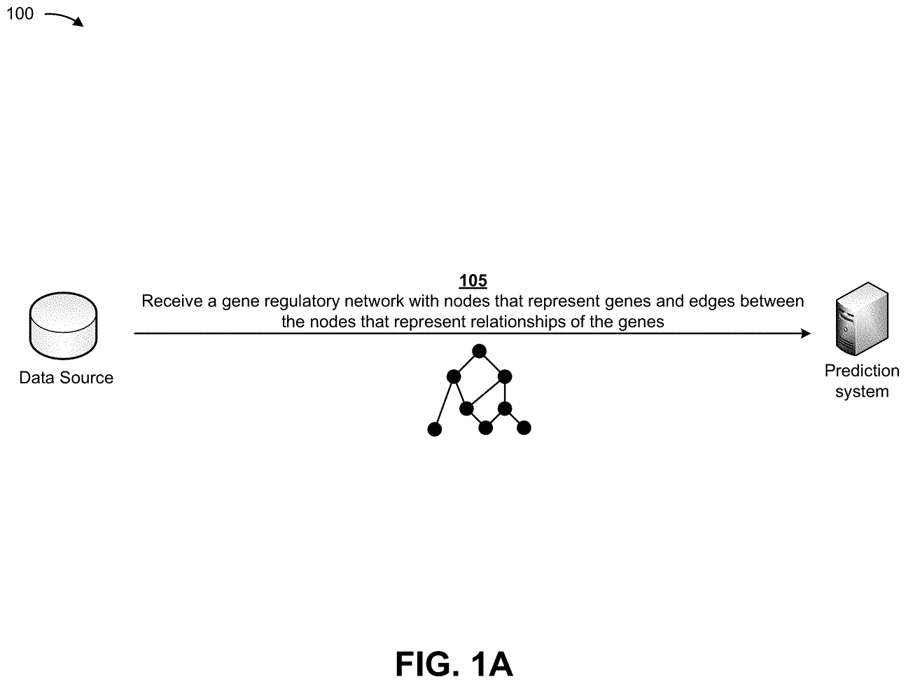

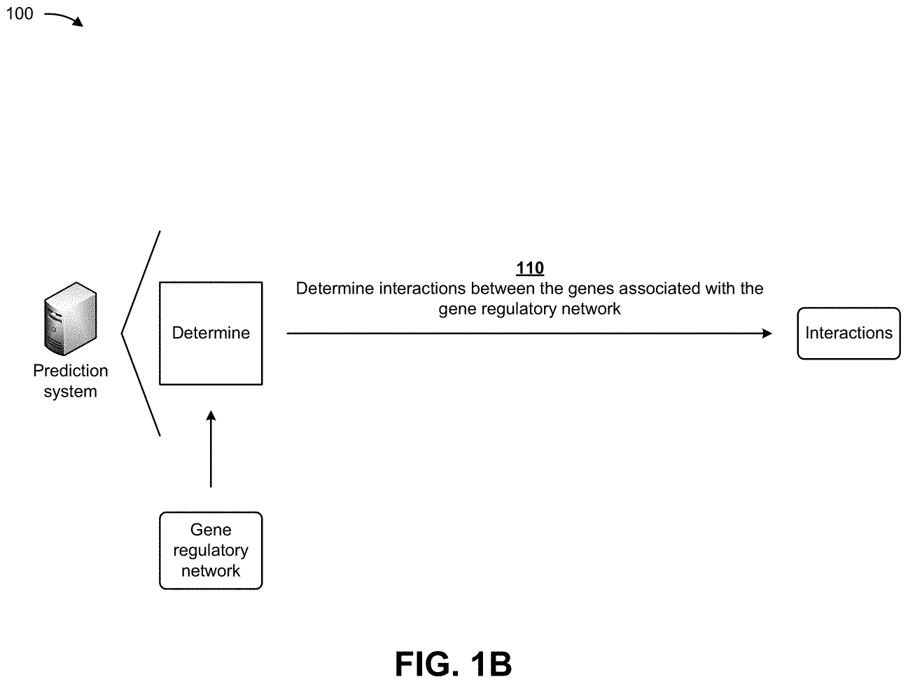

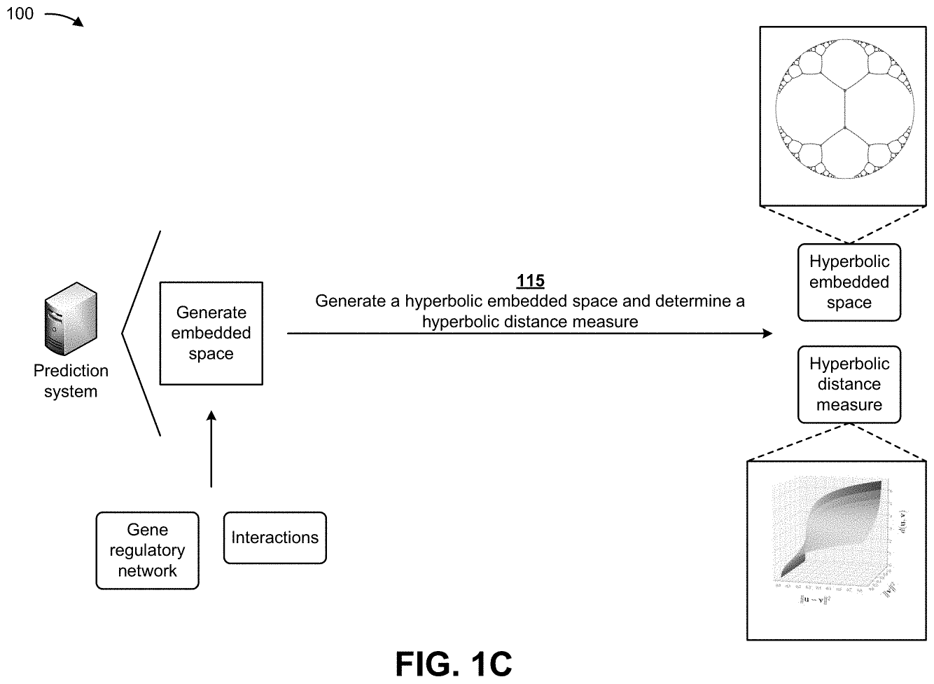

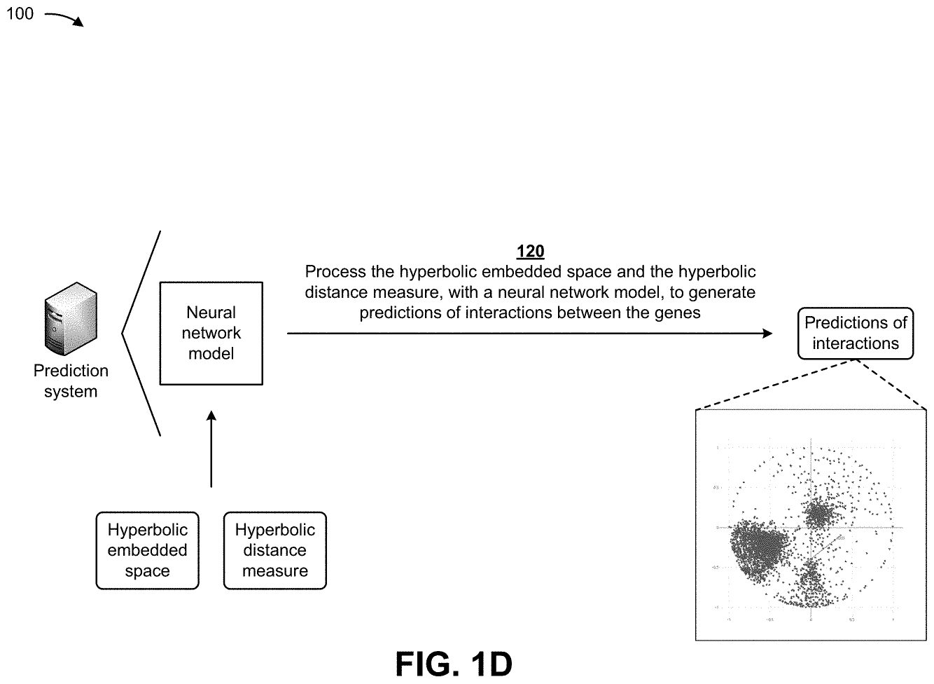

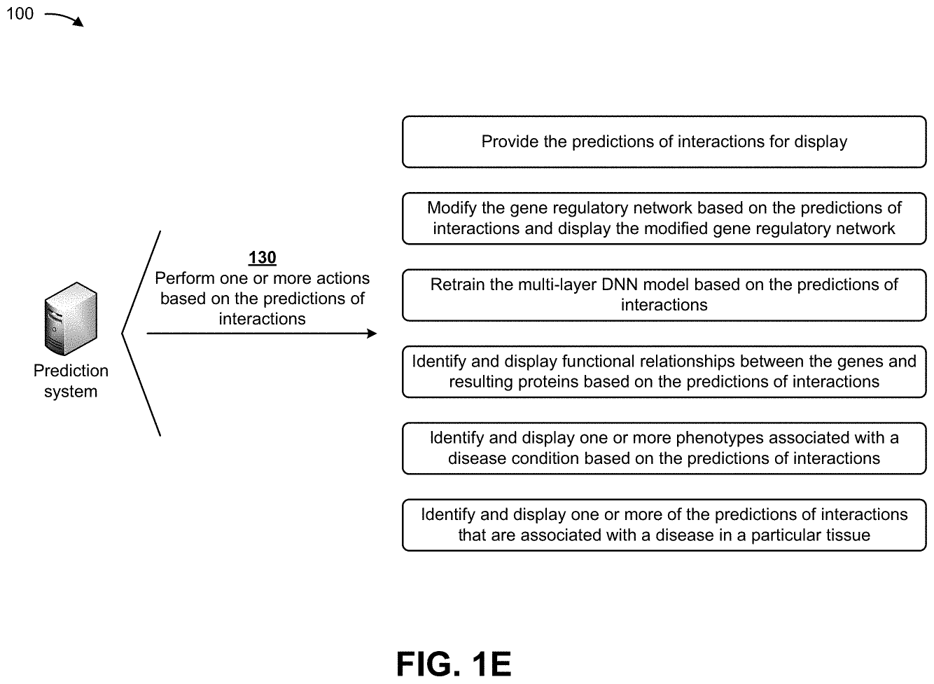

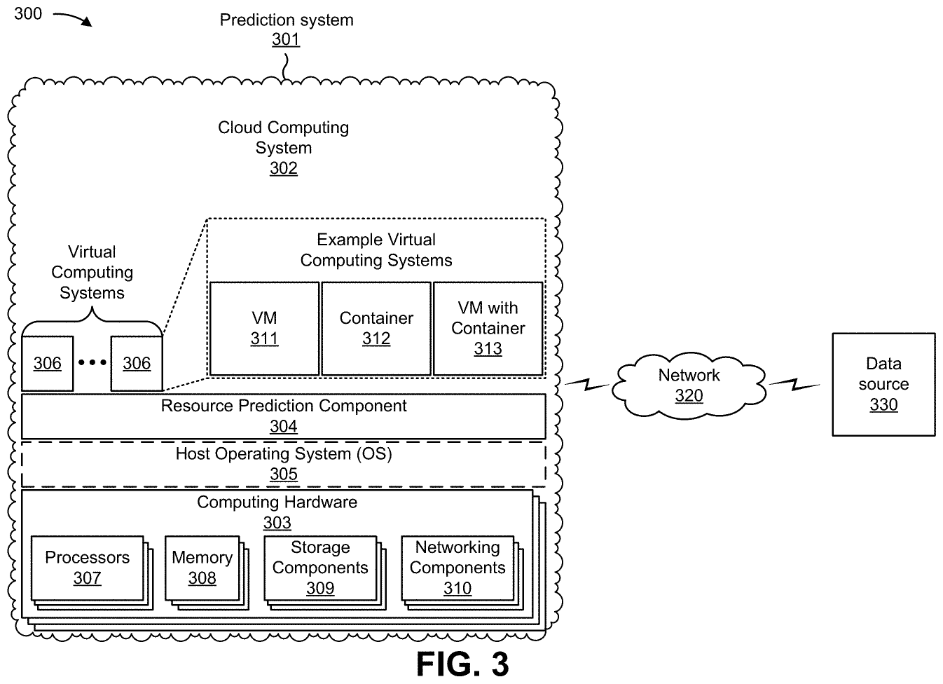

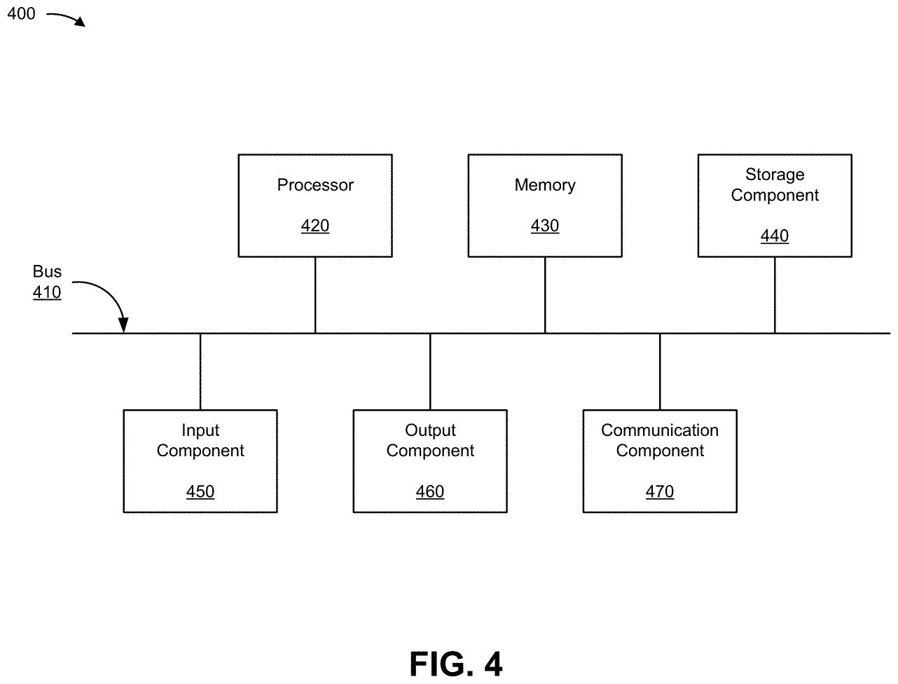

FIGS. 1A-1E are diagrams of an example implementation 100 associated with predicting interactions between genes. As shown in FIGS. 1A-1E, example implementation 100 includes a prediction system and a data source. These devices are described in more detail below in connection with FIG. 3 and FIG. 4. The prediction system and the data source may be connected via a network, such as a wired network (e.g., the Internet or another data network) and/or a wireless network (e.g., a wireless local area network, a wireless wide area network, a cellular network, and/or the like).

As shown in FIG. 1A, and by reference number 105, the prediction system may receive a gene regulatory network from the data source. The gene regulatory network may define functional properties of genomic control programs that are associated with genes (e.g., of one or more animals) and may include representations of the genes interacting to manage molecular functions. For example, the gene regulatory network may include a plurality of nodes that represent genes and a plurality of edges between the nodes that represent relationships of the genes. In some implementations, the prediction system may send a request to the data source for the gene regulatory network and/or the data source may send the gene regulatory network to the prediction system.

Turning to FIG. 1B, and reference number 110, the prediction system may determine interactions between the genes associated with the gene regulatory network. For example, the prediction system may process the gene regulatory network using a graph traversal technique (e.g., a depth-first graph traversal technique and/or a breadth-first graph traversal technique, among other examples) to identify the plurality of nodes (e.g., that represent genes) and/or the plurality of edges (e.g., that represent relationships between the genes). The prediction system may analyze a set of edges, of the plurality of edges, between a set of nodes, of the plurality of nodes, to determine and/or identify the interactions between the genes associated with the set of nodes. For example, the prediction system may determine values associated with the set of edges (e.g., where a value of an edge indicates a strength of a relationship between a first node and a second node connected by the edge) and may determine and/or identify the interactions between the genes associated with the set of nodes based on the values associated with the set of edges (e.g., by averaging the values).

Turning to FIG. 1C, and reference number 115, the prediction system may generate a hyperbolic embedded space that identifies similarities between the genes associated with the gene regulatory network and/or a hierarchy among the genes associated with the gene regulatory network. The hyperbolic embedded space may include a space in which data (e.g., nodes and/or edges of the gene regulatory network) is embedded after dimensionality reduction. In some implementations, the hyperbolic embedded space may be a Poincare embedding space (e.g., based on Poincare ball model) and the prediction system may process the gene regulatory network and/or the interactions (e.g., using a Riemannian optimization technique) to represent the gene regulatory network in the Poincare embedding space.

In some implementations, the prediction system may determine a hyperbolic distance measure (e.g., that indicates a distance between any directly connected nodes within the hyperbolic embedded space) based on the hyperbolic embedded space. For example, when the hyperbolic embedded space is a Poincare embedding space, the prediction system may use a Poincare ball model with a Riemannian metric tensor to determine the hyperbolic distance measure between nodes. Accordingly, for nodes u and v that are included in the Poincare embedding space, the distance measure between u and v may be represented as:

$$d ( u , v ) = ( 1 + 2 \frac{{‖ u - v ‖}^{2}}{( 1 - {‖ u ‖}^{2} ) ( 1 - {‖ v ‖}^{2} )} ) .$$

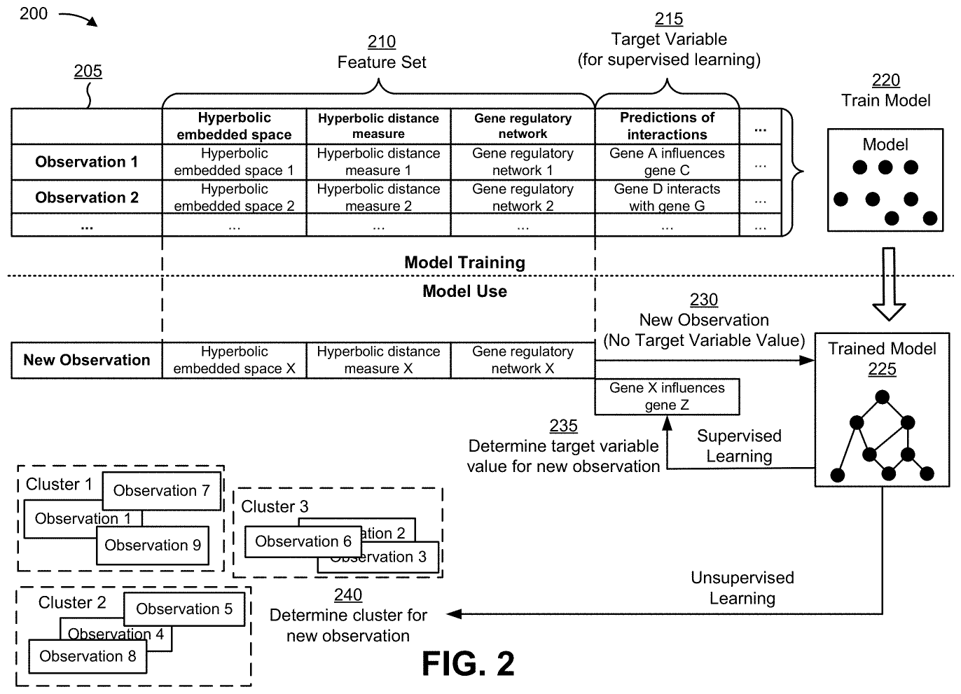

Turning to FIG. 1D, and reference number 120, the prediction system may process the hyperbolic embedded space and/or the hyperbolic distance measure, with a neural network model, to generate predictions of interactions between genes (e.g., of the gene regulatory network). The neural network model may include, for example, a multi-layer deep neural network model. The neural network model may have been trained based on training data that may include example hyperbolic embedded spaces, example hyperbolic distance measures associated with the example hyperbolic embedded spaces, example gene regulatory networks associated with the example hyperbolic embedded spaces, example interactions associated with the example gene regulatory networks, and/or example predictions of interactions associated with the example gene regulatory networks. Using the training data as input to the neural network model, the neural network model may be trained to identify one or more relationships (e.g., between the example hyperbolic embedded spaces, the example hyperbolic distance measures, the example gene regulatory networks, the example interactions, and/or the example predictions of interactions) to generate predictions of interactions between genes. While some implementations are described herein as utilizing a neural network model, any machine learning model may be used in addition to or as an alternative to the neural network model. Accordingly, the neural network model may be trained and/or used in a similar manner to that described below with respect to FIG. 2.

Turning to FIG. 1E, and reference number 130, the prediction system may perform one or more actions based on the predictions of interactions between the genes. In some implementations, the one or more actions may include providing data identifying the predictions of interactions for display. For example, the prediction system may generate and send data identifying the predictions of interactions to a user device, which may cause the data identifying the predictions of interactions to be displayed on a display of the user device. In this way, a user of the user device may obtain information that the user can use to determine whether the gene regulatory network should be updated to indicate some or all of the predictions of interactions.

In some implementations, the one or more actions may include modifying the gene regulatory network based on the predictions of interactions. For example, based on the predictions of interactions, the prediction system may identify a plurality of nodes of the gene regulatory network that are to be connected by one or more new edges and may generate the one or more new edges. The prediction system may cause the one or more new edges to be added to the gene regulatory network, such that the plurality of nodes are connected by the one or more new edges. In this way, the gene regulatory network may be updated to accurately reflect interactions between genes. Further, in some implementations, the prediction system may provide data identifying the modified gene regulatory network for display. For example, the prediction system may generate and send data identifying the modified gene regulatory network to the user device, which may cause the data identifying the predictions of interactions to be displayed on a display of the user device. In this way, a user of the user device may obtain information indicating how the gene regulatory network was updated.

In some implementations, the one or more actions include retraining the neural network model based on the predictions of interactions. For example, the prediction system may use the predictions of interactions as additional training data to retrain and/or update the neural network model. In this way, the prediction system may improve the accuracy of the neural network model, which may improve a speed and/or an efficiency of the neural network model, which conserves computing resources of the prediction system.

In some implementations, the one or more actions includes identifying, based on the predictions of interactions, one or more or more of: data identifying functional relationships between the genes of the gene regulatory network and resulting proteins, data identifying one or more phenotypes associated with a disease condition, and/or data identifying one or more of the predictions of interactions that are associated with a disease, among other examples. For example, the prediction system may identify a set of genes of the gene regulatory network that are associated with the predictions of interactions and may use a protein function prediction algorithm (e.g., a genomic context-based protein function prediction algorithm), based on the set of genes and the predictions of interactions, to identify a set of proteins and functional relationships between the set of genes and the set of proteins. As another example, the prediction system may identify, based on the predictions of interactions, one or more sets of nodes that have a relationship that was not otherwise indicated by the gene regulatory network. The prediction system may determine, based on identifying the set of nodes, that the predictions of interactions are associated with a disease and/or with one or more phenotypes that are associated with the disease. In some implementations, the prediction system may provide the data identifying functional relationships between the genes of the gene regulatory network and resulting proteins, the data identifying one or more phenotypes associated with a disease condition, and/or the data identifying one or more of the predictions of interactions that are associated with a disease, and/or the like, for display (e.g., in a similar manner as that described above). In this way, the prediction system facilitates identifying functional relationships between genes and resulting proteins, as well as insights into underlying biological phenomena that are critical to understanding phenotypes in health and disease conditions (e.g., in particular tissue).

As indicated above, FIGS. 1A-1E are provided as an example. Other examples may differ from what is described with regard to FIGS. 1A-1E. The number and arrangement of devices shown in FIGS. 1A-1E are provided as an example. In practice, there may be additional devices, fewer devices, different devices, or differently arranged devices than those shown in FIGS. 1A-1E. Furthermore, two or more devices shown in FIGS. 1A-1E may be implemented within a single device, or a single device shown in FIGS. 1A-1E may be implemented as multiple, distributed devices. Additionally, or alternatively, a set of devices (e.g., one or more devices) shown in FIGS. 1A-1E may perform one or more functions described as being performed by another set of devices shown in FIGS. 1A-1E.

FIG. 2 is a diagram illustrating an example 200 of training and using a machine learning model, such as a neural network model, in connection with predicting interactions between genes. The machine learning model training and usage described herein may be performed using a machine learning system. The machine learning system may include or may be included in a computing device, a server, a cloud computing environment, or the like, such as the prediction system described in more detail elsewhere herein.

As shown by reference number 205, a machine learning model may be trained using a set of observations. The set of observations may be obtained from training data (e.g., example data), such as data gathered during one or more processes described herein. In some implementations, the machine learning system may receive the set of observations (e.g., as input) from the data source, as described elsewhere herein.

As shown by reference number 210, the set of observations includes a feature set. The feature set may include a set of variables, and a variable may be referred to as a feature. A specific observation may include a set of variable values (or feature values) corresponding to the set of variables. In some implementations, the machine learning system may determine variables for a set of observations and/or variable values for a specific observation based on input received from the data source. For example, the machine learning system may identify a feature set (e.g., one or more features and/or feature values) by extracting the feature set from structured data, by performing natural language processing to extract the feature set from unstructured data, and/or by receiving input from an operator.

As an example, a feature set for a set of observations may include a first feature of a hyperbolic embedding space, a second feature of a hyperbolic distance measure, a third feature of a gene regulatory network, and so on. As shown, for a first observation, the first feature may have a value of hyperbolic embedding space 1, the second feature may have a value of hyperbolic distance measure 1, the third feature may have a value of gene regulatory network 1, and so on. These features and feature values are provided as examples, and may differ in other examples.

As shown by reference number 215, the set of observations may be associated with a target variable. The target variable may represent a variable having a numeric value, may represent a variable having a numeric value that falls within a range of values or has some discrete possible values, may represent a variable that is selectable from one of multiple options (e.g., one of multiples classes, classifications, or labels) and/or may represent a variable having a Boolean value. A target variable may be associated with a target variable value, and a target variable value may be specific to an observation. In example 200, the target variable is predictions of interactions, which has a value of gene A influences gene C for the first observation.

The target variable may represent a value that a machine learning model is being trained to predict, and the feature set may represent the variables that are input to a trained machine learning model to predict a value for the target variable. The set of observations may include target variable values so that the machine learning model can be trained to recognize patterns in the feature set that lead to a target variable value. A machine learning model that is trained to predict a target variable value may be referred to as a supervised learning model.

In some implementations, the machine learning model may be trained on a set of observations that do not include a target variable. This may be referred to as an unsupervised learning model. In this case, the machine learning model may learn patterns from the set of observations without labeling or supervision, and may provide output that indicates such patterns, such as by using clustering and/or association to identify related groups of items within the set of observations.

As shown by reference number 220, the machine learning system may train a machine learning model using the set of observations and using one or more machine learning algorithms, such as a regression algorithm, a decision tree algorithm, a neural network algorithm, a k-nearest neighbor algorithm, a support vector machine algorithm, a multi-layer deep neural network algorithm, or the like. After training, the machine learning system may store the machine learning model as a trained machine learning model 225 to be used to analyze new observations.

As shown by reference number 230, the machine learning system may apply the trained machine learning model 225 to a new observation, such as by receiving a new observation and inputting the new observation to the trained machine learning model 225. As shown, the new observation may include a first feature of hyperbolic embedding space X, a second feature of hyperbolic distance measure X, a third feature of gene regulatory network X, and so on, as an example. The machine learning system may apply the trained machine learning model 225 to the new observation to generate an output (e.g., a result). The type of output may depend on the type of machine learning model and/or the type of machine learning task being performed. For example, the output may include a predicted value of a target variable, such as when supervised learning is employed. Additionally, or alternatively, the output may include information that identifies a cluster to which the new observation belongs and/or information that indicates a degree of similarity between the new observation and one or more other observations, such as when unsupervised learning is employed.

As an example, the trained machine learning model 225 may predict a value of gene X influences gene Z for the target variable of predictions of interactions for the new observation, as shown by reference number 235. Based on this prediction, the machine learning system may provide a first recommendation, may provide output for determination of a first recommendation, may perform a first automated action, and/or may cause a first automated action to be performed (e.g., by instructing another device to perform the automated action), among other examples.

In some implementations, the trained machine learning model 225 may classify (e.g., cluster) the new observation in a cluster, as shown by reference number 240. The observations within a cluster may have a threshold degree of similarity. As an example, if the machine learning system classifies the new observation in a first cluster, then the machine learning system may provide a first recommendation. Additionally, or alternatively, the machine learning system may perform a first automated action and/or may cause a first automated action to be performed (e.g., by instructing another device to perform the automated action) based on classifying the new observation in the first cluster.

In some implementations, the recommendation and/or the automated action associated with the new observation may be based on a target variable value having a particular label (e.g., classification or categorization), may be based on whether a target variable value satisfies one or more threshold (e.g., whether the target variable value is greater than a threshold, is less than a threshold, is equal to a threshold, falls within a range of threshold values, or the like), and/or may be based on a cluster in which the new observation is classified.

In this way, the machine learning system may apply a rigorous and automated process to predicting interactions between genes. The machine learning system enables recognition and/or identification of tens, hundreds, thousands, or millions of features and/or feature values for tens, hundreds, thousands, or millions of observations, thereby increasing accuracy and consistency and reducing delay associated with predicting interactions between genes relative to requiring computing resources to be allocated for tens, hundreds, or thousands of operators to manually predict interactions between genes using the features or feature values.

As indicated above, FIG. 2 is provided as an example. Other examples may differ from what is described in connection with FIG. 2.

FIG. 3 is a diagram of an example environment 300 in which systems and/or methods described herein may be implemented. As shown in FIG. 3, environment 300 may include a prediction system 301, which may include one or more elements of and/or may execute within a cloud computing system 302. The cloud computing system 302 may include one or more elements 303-313, as described in more detail below. As further shown in FIG. 3, environment 300 may include a network 320 and/or a data source 330. Devices and/or elements of environment 300 may interconnect via wired connections and/or wireless connections.

The cloud computing system 302 includes computing hardware 303, a resource management component 304, a host operating system (OS) 305, and/or one or more virtual computing systems 306. The resource management component 304 may perform virtualization (e.g., abstraction) of computing hardware 303 to create the one or more virtual computing systems 306. Using virtualization, the resource management component 304 enables a single computing device (e.g., a computer, a server, and/or the like) to operate like multiple computing devices, such as by creating multiple isolated virtual computing systems 306 from computing hardware 303 of the single computing device. In this way, computing hardware 303 can operate more efficiently, with lower power consumption, higher reliability, higher availability, higher utilization, greater flexibility, and lower cost than using separate computing devices.

Computing hardware 303 includes hardware and corresponding resources from one or more computing devices. For example, computing hardware 303 may include hardware from a single computing device (e.g., a single server) or from multiple computing devices (e.g., multiple servers), such as multiple computing devices in one or more data centers. As shown, computing hardware 303 may include one or more processors 307, one or more memories 308, one or more storage components 309, and/or one or more networking components 310. Examples of a processor, a memory, a storage component, and a networking component (e.g., a communication component) are described elsewhere herein.

The resource management component 304 includes a virtualization application (e.g., executing on hardware, such as computing hardware 303) capable of virtualizing computing hardware 303 to start, stop, and/or manage one or more virtual computing systems 306. For example, the resource management component 304 may include a hypervisor (e.g., a bare-metal or Type 1 hypervisor, a hosted or Type 2 hypervisor, and/or the like) or a virtual machine monitor, such as when the virtual computing systems 306 are virtual machines 311. Additionally, or alternatively, the resource management component 304 may include a container manager, such as when the virtual computing systems 306 are containers 312. In some implementations, the resource management component 304 executes within and/or in coordination with a host operating system 305.

A virtual computing system 306 includes a virtual environment that enables cloud-based execution of operations and/or processes described herein using computing hardware 303. As shown, a virtual computing system 306 may include a virtual machine 311, a container 312, a hybrid environment 313 that includes a virtual machine and a container, and/or the like. A virtual computing system 306 may execute one or more applications using a file system that includes binary files, software libraries, and/or other resources required to execute applications on a guest operating system (e.g., within the virtual computing system 306) or the host operating system 305.

Although the prediction system 301 may include one or more elements 303-313 of the cloud computing system 302, may execute within the cloud computing system 302, and/or may be hosted within the cloud computing system 302, in some implementations, the prediction system 301 may not be cloud-based (e.g., may be implemented outside of a cloud computing system) or may be partially cloud-based. For example, the prediction system 301 may include one or more devices that are not part of the cloud computing system 302, such as device 400 of FIG. 4, which may include a standalone server or another type of computing device. The prediction system 301 may perform one or more operations and/or processes described in more detail elsewhere herein.

Network 320 includes one or more wired and/or wireless networks. For example, network 320 may include a cellular network, a public land mobile network (PLMN), a local area network (LAN), a wide area network (WAN), a private network, the Internet, and/or the like, and/or a combination of these or other types of networks. The network 320 enables communication among the devices of environment 300.

The data source 330 includes one or more devices capable of receiving, generating, storing, processing, and/or providing information associated with providing a gene regulatory network to the prediction system 301, as described elsewhere herein. The data source 330 may include a communication device and/or a computing device. For example, the data source 330 may include a database, a server, a database server, an application server, a client server, a web server, a host server, a proxy server, a virtual server (e.g., executing on computing hardware), a server in a cloud computing system, a device that includes computing hardware used in a cloud computing environment, or a similar type of device. The data source 330 may communicate with one or more other devices of environment 300, as described elsewhere herein.

The number and arrangement of devices and networks shown in FIG. 3 are provided as an example. In practice, there may be additional devices and/or networks, fewer devices and/or networks, different devices and/or networks, or differently arranged devices and/or networks than those shown in FIG. 3. Furthermore, two or more devices shown in FIG. 3 may be implemented within a single device, or a single device shown in FIG. 3 may be implemented as multiple, distributed devices. Additionally, or alternatively, a set of devices (e.g., one or more devices) of environment 300 may perform one or more functions described as being performed by another set of devices of environment 300.

FIG. 4 is a diagram of example components of a device 400, which may correspond to prediction system 301, computing hardware 303, and/or data source 330. In some implementations, prediction system 301, computing hardware 303, and/or data source 330 may include one or more devices 400 and/or one or more components of device 400. As shown in FIG. 4, device 400 may include a bus 410, a processor 420, a memory 430, a storage component 440, an input component 450, an output component 460, and a communication component 470.

Bus 410 includes a component that enables wired and/or wireless communication among the components of device 400. Processor 420 includes a central processing unit, a graphics processing unit, a microprocessor, a controller, a microcontroller, a digital signal processor, a field-programmable gate array, an application-specific integrated circuit, and/or another type of processing component. Processor 420 is implemented in hardware, firmware, or a combination of hardware and software. In some implementations, processor 420 includes one or more processors capable of being programmed to perform a function. Memory 430 includes a random access memory, a read only memory, and/or another type of memory (e.g., a flash memory, a magnetic memory, and/or an optical memory).

Storage component 440 stores information and/or software related to the operation of device 400. For example, storage component 440 may include a hard disk drive, a magnetic disk drive, an optical disk drive, a solid state disk drive, a compact disc, a digital versatile disc, and/or another type of non-transitory computer-readable medium. Input component 450 enables device 400 to receive input, such as user input and/or sensed inputs. For example, input component 450 may include a touch screen, a keyboard, a keypad, a mouse, a button, a microphone, a switch, a sensor, a global positioning system component, an accelerometer, a gyroscope, and/or an actuator. Output component 460 enables device 400 to provide output, such as via a display, a speaker, and/or one or more light-emitting diodes. Communication component 470 enables device 400 to communicate with other devices, such as via a wired connection and/or a wireless connection. For example, communication component 470 may include a receiver, a transmitter, a transceiver, a modem, a network interface card, and/or an antenna.

Device 400 may perform one or more processes described herein. For example, a non-transitory computer-readable medium (e.g., memory 430 and/or storage component 440) may store a set of instructions (e.g., one or more instructions, code, software code, and/or program code) for execution by processor 420. Processor 420 may execute the set of instructions to perform one or more processes described herein. In some implementations, execution of the set of instructions, by one or more processors 420, causes the one or more processors 420 and/or the device 400 to perform one or more processes described herein. In some implementations, hardwired circuitry may be used instead of or in combination with the instructions to perform one or more processes described herein. Thus, implementations described herein are not limited to any specific combination of hardware circuitry and software.

The number and arrangement of components shown in FIG. 4 are provided as an example. Device 400 may include additional components, fewer components, different components, or differently arranged components than those shown in FIG. 4. Additionally, or alternatively, a set of components (e.g., one or more components) of device 400 may perform one or more functions described as being performed by another set of components of device 400.

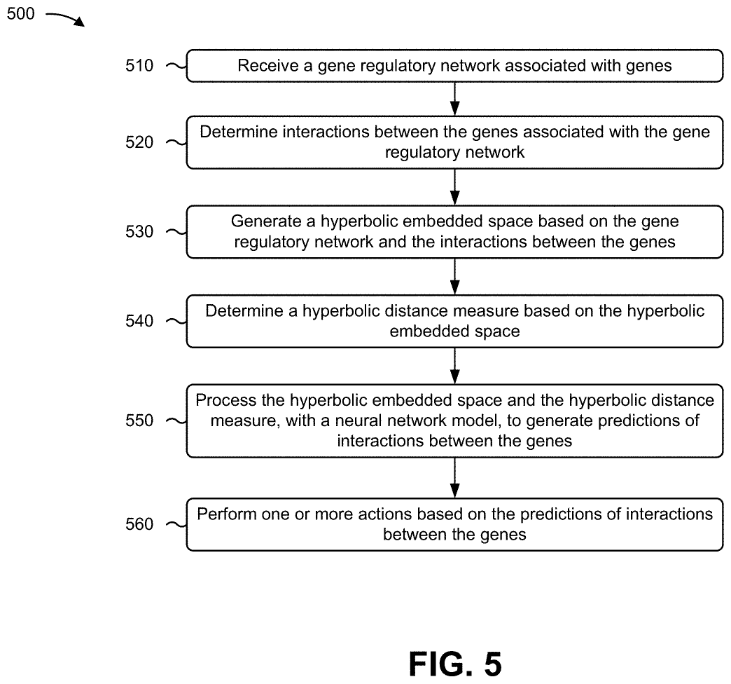

FIG. 5 is a flowchart of an example process 500 associated with predicting interactions between genes. In some implementations, one or more process blocks of FIG. 5 may be performed by a device (e.g., prediction system 301). In some implementations, one or more process blocks of FIG. 5 may be performed by another device or a group of devices separate from or including the device, such as a data source (e.g., data source 330). Additionally, or alternatively, one or more process blocks of FIG. 5 may be performed by one or more components of device 400, such as processor 420, memory 430, storage component 440, input component 450, output component 460, and/or communication component 470.

As shown in FIG. 5, process 500 may include receiving a gene regulatory network associated with genes (block 510). For example, the device may receive a gene regulatory network associated with genes, as described above. In some implementations, the gene regulatory network defines functional properties of genomic control programs, and includes representations of the genes interacting to manage molecular functions.

As further shown in FIG. 5, process 500 may include determining interactions between the genes associated with the gene regulatory network (block 520). For example, the device may determine interactions between the genes associated with the gene regulatory network, as described above.

As further shown in FIG. 5, process 500 may include generating a hyperbolic embedded space based on the gene regulatory network and the interactions between the genes (block 530). For example, the device may generate a hyperbolic embedded space based on the gene regulatory network and the interactions between the genes, as described above. In some implementations, the hyperbolic embedded space is a Poincare embedding space based on a Poincare ball model. The hyperbolic embedded space may identify similarities between the genes associated with the gene regulatory network and a hierarchy among the genes associated with the gene regulatory network.

As further shown in FIG. 5, process 500 may include determining a hyperbolic distance measure based on the hyperbolic embedded space (block 540). For example, the device may determine a hyperbolic distance measure based on the hyperbolic embedded space, as described above. In some implementations, determining the hyperbolic distance measure includes utilizing a Poincare ball model with a Riemannian metric tensor to determine the hyperbolic distance measure based on the hyperbolic embedded space.

As further shown in FIG. 5, process 500 may include processing the hyperbolic embedded space and the hyperbolic distance measure, with a neural network model, to generate predictions of interactions between the genes (block 550). For example, the device may process the hyperbolic embedded space and the hyperbolic distance measure, with a neural network model, to generate predictions of interactions between the genes, as described above. In some implementations, the neural network model is a multi-layer deep neural network model.

As further shown in FIG. 5, process 500 may include performing one or more actions based on the predictions of interactions between the genes (block 560). For example, the device may perform one or more actions based on the predictions of interactions between the genes, as described above. In some implementations, performing the one or more actions comprises includes determining data identifying one or more of functional relationships between the genes and resulting proteins based on the predictions of interactions, a phenotype associated with a disease condition based on the predictions of interactions, or one or more of the predictions of interactions that are associated with a disease, and providing, to a user device, the data identifying the one or more of the functional relationships, the phenotype, or the one or more of the predictions of interactions.

Process 500 may include additional implementations, such as any single implementation or any combination of implementations described below and/or in connection with one or more other processes described elsewhere herein.

In a first implementation, the gene regulatory network includes nodes that represent the genes and edges between the nodes that represent relationships of the genes.

In a second implementation, alone or in combination with the first implementation, the hyperbolic embedded space includes a space in which data is embedded after dimensionality reduction.

In a third implementation, alone or in combination with one or more of the first and second implementations, the neural network model includes a multi-layer deep neural network model.

In a fourth implementation, alone or in combination with one or more of the first through third implementations, performing the one or more actions comprises one or more of identifying, and providing for display, data identifying functional relationships between the genes and resulting proteins based on the predictions of interactions, identifying, and providing for display, data identifying one or more phenotypes associated with a disease condition based on the predictions of interactions, or identifying, and providing for display, data identifying one or more of the predictions of interactions that are associated with a disease.

In a fifth implementation, alone or in combination with one or more of the first through fourth implementations, performing the one or more actions comprises one or more of providing data identifying the predictions of interactions for display, or retraining the neural network model based on the predictions of interactions.

In a sixth implementation, alone or in combination with one or more of the first through fifth implementations, performing the one or more actions comprises modifying the gene regulatory network based on the predictions of interactions and to generate a modified gene regulatory network, and providing data identifying the modified gene regulatory network for display.

In a seventh implementation, alone or in combination with one or more of the first through sixth implementations, generating the hyperbolic embedded space based on the gene regulatory network includes processing the gene regulatory network using a Riemannian optimization technique to generate the hyperbolic embedded space.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

The foregoing disclosure provides illustration and description, but is not intended to be exhaustive or to limit the implementations to the precise form disclosed. Modifications may be made in light of the above disclosure or may be acquired from practice of the implementations.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code - it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, etc., depending on the context.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method, comprising:
receiving, by a device, a gene regulatory network associated with genes;
determining, by the device, interactions between the genes associated with the gene regulatory network;
generating, by the device, a hyperbolic embedded space based on the gene regulatory network and the interactions between the genes;
determining, by the device, a hyperbolic distance measure based on the hyperbolic embedded space;
processing, by the device, the hyperbolic embedded space and the hyperbolic distance measure, with a neural network model, to generate predictions of interactions between the genes; and
performing, by the device, one or more actions based on the predictions of interactions between the genes.

2. The method of claim 1, wherein the gene regulatory network includes nodes that represent the genes and edges between the nodes that represent relationships of the genes.

3. The method of claim 1, wherein the hyperbolic embedded space includes a space in which data is embedded after dimensionality reduction.

4. The method of claim 1, wherein the neural network model includes a multi-layer deep neural network model.

5. The method of claim 1, wherein performing the one or more actions comprises one or more of:
identifying, and providing for display, data identifying functional relationships between the genes and resulting proteins based on the predictions of interactions;
identifying, and providing for display, data identifying one or more phenotypes associated with a disease condition based on the predictions of interactions; or
identifying, and providing for display, data identifying one or more of the predictions of interactions that are associated with a disease.

6. The method of claim 1, wherein performing the one or more actions comprises one or more of:
providing data identifying the predictions of interactions for display; or
retraining the neural network model based on the predictions of interactions.

7. The method of claim 1, wherein performing the one or more actions comprises:
modifying the gene regulatory network based on the predictions of interactions and to generate a modified gene regulatory network; and
providing data identifying the modified gene regulatory network for display.

8. A device, comprising:
one or more memories; and
one or more processors, communicatively coupled to the one or more memories, configured to:
receive a gene regulatory network associated with genes;
generate a hyperbolic embedded space based on the gene regulatory network;
determine a hyperbolic distance measure based on the hyperbolic embedded space;
process the hyperbolic embedded space and the hyperbolic distance measure, with a neural network model, to generate predictions of interactions between the genes;
modify the gene regulatory network based on the predictions of interactions and to generate a modified gene regulatory network; and
provide data identifying the modified gene regulatory network for display.

9. The device of claim 8, wherein the gene regulatory network defines functional properties of genomic control programs, and includes representations of the genes interacting to manage molecular functions.

10. The device of claim 8, wherein the neural network model is a multi-layer deep neural network model.

11. The device of claim 8, wherein the one or more processors, when generating the hyperbolic embedded space, are configured to:
process the gene regulatory network using a Riemannian optimization technique to generate the hyperbolic embedded space.

12. The device of claim 8, wherein the one or more processors, when determining the hyperbolic distance measure, are configured to:
utilize a Poincaré ball model with a Riemannian metric tensor to determine the hyperbolic distance measure based on the hyperbolic embedded space.

13. The device of claim 8, wherein the hyperbolic embedded space identifies similarities between the genes associated with the gene regulatory network and a hierarchy among the genes associated with the gene regulatory network.

14. The device of claim 8, wherein the one or more processors are further configured to:
determine data identifying one or more of:
functional relationships between the genes and resulting proteins based on the predictions of interactions,
a phenotype associated with a disease condition based on the predictions of interactions, or
one or more of the predictions of interactions that are associated with a disease; and
provide, to a user device, the data identifying the one or more of the functional relationships, the phenotype, or the one or more of the predictions of interactions.

15. A non-transitory computer-readable medium storing a set of instructions, the set of instructions comprising:
one or more instructions that, when executed by one or more processors of a device, cause the device to:
receive a gene regulatory network,
wherein the gene regulatory network includes nodes that represent genes and edges between the nodes that represent relationships of the genes;
generate a hyperbolic embedded space based on the gene regulatory network,
wherein the hyperbolic embedded space includes a space in which data is embedded after dimensionality reduction;
determine a hyperbolic distance measure based on the hyperbolic embedded space;
process the hyperbolic embedded space and the hyperbolic distance measure, with a multi-layer deep neural network model, to generate predictions of interactions between the genes; and
perform one or more actions based on the predictions of interactions between the genes.

16. The non-transitory computer-readable medium of claim 15, wherein the one or more instructions, that cause the device to perform the one or more actions, cause the device to:
identify, and provide for display, data identifying functional relationships between the genes and resulting proteins based on the predictions of interactions;
identify, and provide for display, data identifying one or more phenotypes associated with a disease condition based on the predictions of interactions;
identify, and provide for display, data identifying one or more of the predictions of interactions that are associated with a disease;
provide data identifying the predictions of interactions for display; or
retrain the multi-layer deep neural network model based on the predictions of interactions.

17. The non-transitory computer-readable medium of claim 15, wherein the one or more instructions, that cause the device to perform the one or more actions, cause the device to:
modify the gene regulatory network based on the predictions of interactions and to generate a modified gene regulatory network; and
provide, to a user device, data identifying the modified gene regulatory network.

18. The non-transitory computer-readable medium of claim 15, wherein the hyperbolic embedded space is a Poincaré embedding space based on a Poincaré ball model.

19. The non-transitory computer-readable medium of claim 15, wherein the one or more instructions, that cause the device to generate the hyperbolic embedded space based on the gene regulatory network, cause the device to:
process the gene regulatory network using a Riemannian optimization technique to generate the hyperbolic embedded space.

20. The non-transitory computer-readable medium of claim 15, wherein the one or more instructions, that cause the device to determine the hyperbolic distance measure based on the hyperbolic embedded space, cause the device to:
utilize a Poincaré ball model with a Riemannian metric tensor to determine the hyperbolic distance measure based on the hyperbolic embedded space.
